(19) United States
(12) Patent Application Publication
(10) Pub. No.: US 2024/0188592 A1
(43) Pub. Date: Jun. 13, 2024

(54) USING OXYGEN IN NANOBUBBLES TO IMPROVE CHARACTERISTICS OF POULTRY

(71) Applicants: Sameer H. Israni, Darien, IL (US); Balazs Hunek, Western Springs, IL (US)

(72) Inventors: Sameer H. Israni, Darien, IL (US); Balazs Hunek, Western Springs, IL (US)

(21) Appl. No.: 18/064,376

(22) Filed: Dec. 12, 2022

(51) Int. Cl. A23K 50/75; A23K 20/20; B01F 23/2373; B82Y 40/00

(52) U.S. Cl. CPC A23K 50/75; A23K 20/20; B01F 23/2373; B82Y 40/00

(57) ABSTRACT

Administration to chickens or other poultry of water containing nanobubbles that contain oxygen reduces the incidence of woody breast disorder in the chicken or other poultry.

### FIELD OF THE INVENTION

The present invention relates to treatment of poultry, especially chickens, to improve the characteristics of the chicken from a time at or after hatching of the egg and thereafter as the chick grows to an adult grown chicken. The characteristics that may be improved include reduced or eliminated incidence of a condition known as Woody Breast in the hatched, grown chicken.

### BACKGROUND OF THE INVENTION

A large proportion of modern chickens that are grown to be sources of their meat to be consumed, especially the ones grown in the US, are grown to relatively large sizes (heavier than 6 pounds). The large size is believed to be desirable as it provides breast meat in relatively large quantities per chicken. Chickens grown for their meat are called “broilers”. Chickens grown to larger sizes (heavier than 6 pounds) for their meat are sometimes referred to as “big birds”.

However, chickens of these large sizes, especially heavier than 6 pounds, are often found to have a condition called “Woody Breast” or “Wooden Breast” (referred to herein as Woody Breast or WB). In this condition the breast muscles (which provide the breast meat) have a very hard texture and lower nutritional value. These characteristics substantially reduce the value of the breast meat. It has been estimated that on average 5-20% of big birds (6 pounds or more in weight at the time of slaughter) suffer from Woody Breast, resulting in undesirable overall losses to the chicken growers. Woody Breast is exhibited in the live chicken, before any processing or cooking of the chicken for its meat, and is not prevented or reduced by any processing or cooking of the breast meat. The search has been ongoing for a solution for treating or avoiding Woody Breast condition, other than reducing the size to which the chickens are grown, which itself would reduce their overall value.

### BRIEF SUMMARY OF THE INVENTION

The present invention comprises a method of reducing or eliminating the incidence of woody breast in poultry, preferably in a chicken, comprising administering enterally to the poultry water comprising in the water nanobubbles which comprise oxygen. In preferred embodiments of the present invention, the nanobubbles comprise at least 21 vol. % oxygen, preferably 50 vol. % to 100 vol. % oxygen, more preferably 90 vol. % to 100 vol. % oxygen, and yet more preferably at least 99.0 vol. % oxygen.

In other preferred embodiments of the present invention, the average diameter of said nanobubbles is 10 to 1000 nanometers, preferably 10 to 400 nanometers, more preferably 30 to 200 nanometers, and yet more preferably 70 to 130 nanometers.

In yet other preferred embodiments of the present invention, the water administered to the chicken or other poultry comprises at least 100 million of said nanobubbles per milliliter of water, preferably at least 200 million of said nanobubbles per milliliter of water, and more preferably at least 500 million of said nanobubbles per milliliter of water.

In another preferred embodiment of the present invention, the aggregate amount of oxygen in said nanobubbles and dissolved in the water administered to the chicken or other poultry is 20 to 150 milligrams of oxygen per liter of said water, preferably 35 to 100 milligrams of oxygen per liter of said water, and more preferably 60 to 100 milligrams of oxygen per liter of said water.

In the present invention, 50 vol. % to 100 vol. % and preferably 90 vol. % to 100 vol. % of the water and more preferably at least 95 vol. % or even 99 vol. % to 100 vol. % of the water that is made available to the chicken or other poultry to drink contains oxygen-containing nanobubbles as described herein. The provision for administration of water containing oxygen-containing nanobubbles as described herein preferably begins upon hatching of the chicken or other poultry from its egg, but can begin up to 2 days and even up to 2 weeks from hatching. It is also preferred that said water containing nanobubbles that is administered enterally to the chicken is maintained at a pressure of up to 25 psig until the chicken ingests it.

Advantageously, in the present invention the nanobubbles that are administered in water to the chicken or other poultry are absorbed through the gastrointestinal system of the bird into the circulatory system of the bird.

As used herein, “poultry” means chicken, turkey, duck, and goose. Also, “poultry” is used herein to refer to a single bird, or to a plurality of birds, depending on the context in which the word “poultry” is used.

### BRIEF DESCRIPTION OF THE DRAWINGS

FIG. 1 is a flowsheet showing apparatus useful in performing the present invention.

FIGS. 2 and 3 are bar graphs of nanobubble concentrations in water.

FIG. 4 is a bar graph of scores of the extent of woody breast for groups of chickens.

FIG. 5 is a bar graph of scores of the extent of woody breast for other groups of chickens.

### DETAILED DESCRIPTION OF THE INVENTION

This invention mitigates WB in chickens by feeding them drinking water that contains oxygen nanobubbles. Nanobubbles are bubbles less than 1 micron is diameter and preferably less than 400 microns in diameter, and more preferably less than 250 microns in size. The nanobubbles of a size within a range as described herein, and containing oxygen at a concentration within a range as described herein, and maintained at a pressure within a range as described herein, fed to the chickens as described herein, have been found to be successful in significantly reducing WB incidences.

Forming in water the nanobubbles having the desired characteristics described herein, involves the following:

Oxygen is added into the drinking water that will be fed to the chickens to form oxygen-enriched nanobubbles (by which is meant nanobubbles having oxygen in a content higher than the oxygen content of atmospheric air.) The drinking water is typically well water or city water, but can be from any source of potable water.

Any of several known methods can be employed to generate in the water the desired oxygen-enriched nanobubbles having the desired oxygen content, size, and concentration. There exist multiple methods of generating oxygen-enriched nanobubbles, e.g. (1) dissolving oxygen gas into water at high pressure, then de-pressurizing the water at rate that is controlled to ensure that nanobubbles of oxygen are generated during the de-pressurization; (2) using a venturi based gas liquid mixing device that controls the size and number of bubbles generated; or (3) passing oxygen-containing gas through a nanoporous membrane (with or without a surface coating) to create very small bubbles in the liquid on the discharge side of the membrane. For the purposes of this invention, the technique (1) of pressure-dissolution described above was used and is preferred.

The concentration of oxygen in the gas used to make the nanobubbles should be higher than in air, preferably at least 90 vol. % (with the balance remaining being primarily other inert gases like nitrogen and argon), and more preferably should be more than 99.9 vol. %, and most preferably should be more than 99.99% purity oxygen.

The mean diameter of stable nanobubbles generated should be between 30 to 200 nanometers (nm), as measured by NanoSight analysis (NTA, available commercially through Malvern Panalytical). Preferably the mean diameter should be between 70 to 130 nm.

The amount of oxygen nanobubbles generated should be at least 100 million nanobubbles per milliliter (ml) of the water in which the nanobubbles are formed, preferably above 200 million bubbles per ml, and most preferably above 500 million bubbles per ml of the water.

A useful way to measure the number generated of oxygen-enriched nanobubbles is described below. If NTA is used as the method of analysis then the first step is to measure the concentration of nanobubbles in the original drinking water before it is oxygenated. This measured concentration is referred to as Nᵢₙᵢₜᵢₐₗ. This Nᵢₙᵢₜᵢₐₗ includes nanobubbles that contain air air, and may also contain other solid nanoparticles that do not provide any benefits for the purposes of this invention. Then the drinking water is oxygenated with oxygen-enriched nanobubbles as described herein, and a sample of this water is analyzed by NTA. The measured concentration of nanobubbles in this sample is N_{final}. The actual number of generated oxygen-enriched nanobubbles is then calculated as N_{O2NB}=N_{final}−Nᵢₙᵢₜᵢₐₗ. For the practice of the present invention, this N_{O2NB} should be at least 100 million oxygen-enriched nanobubbles per ml of water containing the nanobubbles, preferably above 200 million oxygen-enriched nanobubbles per ml, and most preferably above 500 million oxygen-enriched nanobubbles per ml of drinking water.

The dissolved oxygen (DO) measured in the drinking water should be between 20 to 45 ppm of oxygen. This is measured using a DO probe by taking a sample of the drinking water with the generated oxygen-enriched nanobubbles and measuring the DO at atmospheric pressure. DO probes only measure the dissolved oxygen portion of the oxygen added to the drinking water. DO probes do not measure the amount of oxygen added to generate the oxygen nanobubbles.

The water that is used to administer the oxygen-enriched nanobubbles should be oxygenated to a level between 20 to 150 mg of total added oxygen per liter of water and more preferably between 35 to 100 mg/l, most preferably between 60 to 100 mg/l. The total added oxygen is measured as the total oxygen present in the water which includes the dissolved oxygen plus the oxygen present in the oxygen-enriched nanobubbles. The amount of oxygen present in the oxygen-enriched nanobubbles can be measured by using a sodium sulfite titration methodology. Throughout the process of generation of oxygen-enriched nanobubbles and even after the generation of the desired quantity of oxygen-enriched nanobubbles, the water containing the nanobubbles should be maintained under higher than atmospheric pressure.

Techniques for forming nanobubbles in water, wherein the characteristics are as desired herein such as the number and size of the nanobubbles in the water, are known in the technical literature such as U.S. Pat. No. 10,293,309 and the U.S. patent application published under publication number US2007/0286795A1. Devices for forming nanobubbles in water, that are useful in performing the present invention, are known and available commercially.

Several prior art documents specify adding chemical components that enhance the stability of the nanobubbles in the water. For example, the U.S. patent application published under publication number US2007/0286795A1 specifies increasing the salinity of the water up to 3.5%. However, for the purpose of drinking water, these additional chemicals can cause adverse effects on birds. It is highly desirable to avoid adding any additional chemicals to the drinking water besides the oxygen rich gas. An important step in the current invention is to maintain slight pressure on the nanobubble containing drinking water. In the absence of chemicals to enhance the stability of the nanobubbles, this slight positive pressure maintains the stability of the nanobubbles, providing the Woody Breast reduction efficacy. While not preferred, the water that contains the nanobubbles can contain other substances, such as medications, nutrients etc. provided that any substances present in the water do not adversely affect the health of the chicken.

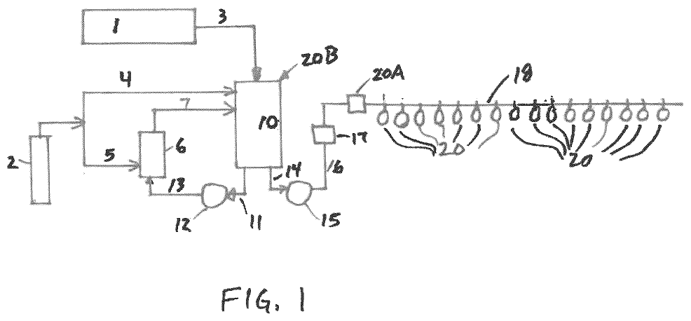

The water that contains the nanobubbles should be at a temperature of no more than 5 to 10 degrees F. above room temperature, the better to protect against the oxygen being released from the water. Preferably the temperature of the water is 35 to 125 degrees F. more preferably 50 to 100 degrees F. Accordingly, provision can be included in the apparatus described with respect to FIG. 1 to cool or heat the water as needed in order to prevent the temperature of the water from increasing undesirably.

The drinking water containing oxygen-enriched nanobubbles as described herein is administered by any of several possible methodologies. For instance, a human operator or handler can insert into each chicken's mouth the end of a dropper or syringe filled with the water, and feed the water out of the end of the dropper or syringe into the chicken's mouth or throat.

A preferable mode of administering to the chicken the drinking water containing oxygen-enriched nanobubbles is to provide the water in a way that the chicken can drink the water entirely by its own actions, whenever it wants to drink water, without needing intervention of another person (other than, possibly, demonstration to the chicken of how to gain access to the water, when the chicken is first put in proximity to the supply of the water containing the oxygen-enriched nanobubbles). Preferably, in this arrangement, all of the water that is available for the chicken to drink contains oxygen-enriched nanobubbles as described herein. However, benefits of the present invention are available (though perhaps to a smaller extent) if 5 vol. % or more of the water that is available for the chicken to drink contains the oxygen-containing nanobubbles as described herein; a greater benefit is realized if at least 50 vol % and more preferably 90 vol. % of the water that is available for the chicken to drink contains the oxygen-containing nanobubbles, and even more preferably at least 99 vol. % of the water that is available for the chicken to drink contains the oxygen-containing nanobubbles.

A preferred apparatus with which the present invention may be performed is shown in FIG. 1, in which can be seen source 1 of water to be oxygenated with the nanobubbles, and source 2 of oxygen which is preferably a cylinder or similar dispenser that contains oxygen that is preferably at a purity of at least 95 vol. % and preferably at least 99 vol. %. Water flows through line 3 from water source 1 to tank 10. Oxygen flows through line 4 from oxygen source 2 to tank 10, and through line 5 from oxygen source 2 to nanobubble generator 6 which can be any commercially available device able to generate nanobubbles in the water to the extent and with the characteristics desired as described herein. Water containing nanobubbles as described herein flows through line 7 from nanobubble generator 6 into tank 10. Water containing nanobubbles as described herein can be recycled from tank 10 through line 11 and recirculating pump 12 into line 13 which feeds the water into nanobubble generator 6. Water containing the nanobubbles as described herein is fed out of tank 10 in line 14 through pump 15 into line 16 which conveys the water containing the oxygen-containing nanobubbles to one or more devices 18 such as nipples 20 which hold the water containing the oxygen-enriched nanobubbles and which do not permit water to pass out of the device 18 or the nipples 20 except when the chicken taps the device (i.e. a nipple 20) with its beak. Optional cooler 17 can be placed in line 16 to maintain the temperature of the water in line 16 at a level that is not so high that evolution of oxygen from the water occurs. Alternatively, the cooler 17 could be placed inside tank 10, in line 13 or other similar places. Preferably, pressurized nipple drinkers on the vessel containing the water should be used to ensure that the water remains pressurized until a few seconds before the chicken drinks it. The pressure on the drinking water containing the oxygen-containing nanobubbles at this stage should be above atmospheric pressure, preferably at least 1 psig and up to 25 psig. Typically this is achieved by using commercially available nipple type drinkers, that include a component 20A which maintains the water in a pressurized state. Pressure on the water that contains the oxygen-containing nanobubbles can also be maintained by a suitable component 20B that applies pressure to the water in tank 10. Pressure on the water line can also be maintained by the pump 15.

The birds tap on one of several nipples 20 when they want water. The tapping temporarily opens the nipple 20 allowing one or more drops of water containing oxygen-enriched nanobubbles to come out and be consumed by the chicken. An alternative apparatus includes a bowl that is open to the atmosphere and that contains water containing oxygen-enriched nanobubbles.

The oxygen-enriched nanobubbles in the drinking water should be replenished at regular intervals to ensure that the concentration does not drop below the specified value of 100 million bubbles per ml of water. Preferably, the replenishment is carried out at least every 12 hours. This means that the equipment for oxygenating the water with oxygen-enriched nanobubbles should be run at least every 12 hours to make sure that the level of oxygen-enriched nanobubbles present in the drinking water remains above 100 million bubbles per ml, preferably above 200 million bubbles per ml, and most preferably above 500 million bubbles per ml of drinking water.

The chicken will benefit from drinking the oxygen-enriched drinking water for even small periods of time at any stage of their growth. Preferably, the drinking water with oxygen-enriched nanobubbles should be fed to the chickens either immediately upon hatching or at the most starting from no longer than 2 weeks, more preferably no longer than 2 days, after hatching. Typically, the drinking water is available to the chickens on demand, i.e. whenever they want without restriction. From the start point, the drinking water containing oxygen-enriched nanobubbles can continue to be fed to the chicken everyday for different periods of time, preferably at a minimum for one week from the start of the administration, but most preferably for the entire growing period until the chickens are fully grown. Typically commercial broilers are grown for a period of 6 to 9 weeks from hatch. Thus the total period during which the chicken is fed water containing oxygen-enriched nanobubbles should be between 1 week up to the entire lifetime after hatching.

The main benefit of the above methodology is a 50 to 100% reduction in WB scores for broiler chicken that drink the water containing oxygen-enriched nanobubbles. An example of the benefit is described in the Examples below. Other potential benefits of the technology include better health, lower mortality, higher weight gains, more efficient feed conversion, and better meat quality. This methodology can also be employed with any other living species in addition to broiler chickens.

The WB benefits apply the most to broilers that are going to be grown to a slaughter weight of 6 pounds or higher, and more preferably 6.5 pounds or higher. Only certain genetic lines of birds are chosen to be grown to be larger than 6.5 pounds in weight. Other benefits apply to all category of broiler and potentially all living species.

### EXAMPLES

Oxygen nanobubbles were generated in tap water using pure oxygen gas and a pressure-dissolution based nanobubble generator. No NB stability enhancing chemicals or components we added to the water. The concentration of oxygen nanobubbles generated was approximately 100 million bubbles per ml of tap water. This concentration was measured via NTA after subtracting for the initial nanobubble concentration in the tap water.

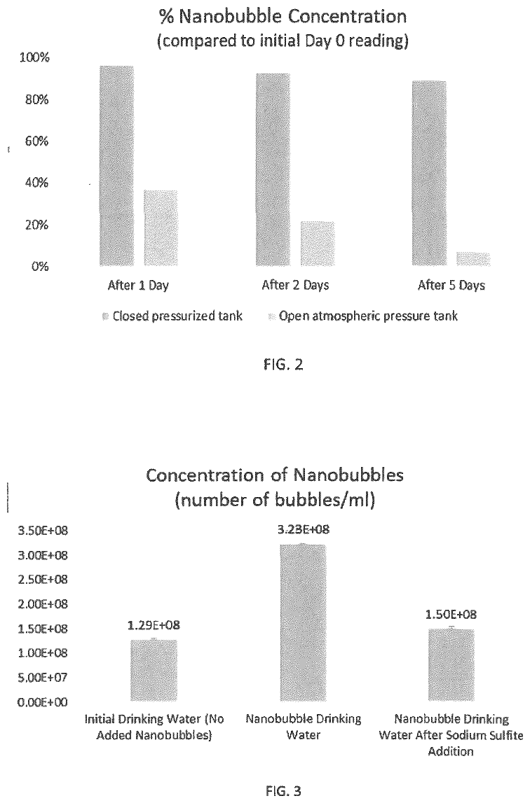

The oxygen nanobubble water was split into two halves. One half was kept in a closed tank that had a very slight headspace pressure of approximately 1 psig. The other half was kept in an open vessel at atmospheric pressure. Samples were taken from these two different tanks at day 1, 2 and 5 after start of storage. These samples were analyzed for nanobubble concentration using NTA. The graph in FIG. 2 shows the concentration of nanobubbles expressed as % compared to the initial nanobubble concentration. The oxygen-containing nanobubbles stored in the closed vessel under slight pressure lasted much longer than the nanobubbles stored in an open tank at atmospheric pressure. This shows the importance of (1) keeping the water in which oxygen-containing nanobubbles had been generated under at least slight pressure in a closed tank and pipeline until the chickens drink the water and (2) regenerating the oxygen-containing nanobubbles in the water at least every 12 hours to ensure that the chickens are getting the required concentration of oxygen-containing nanobubbles.

The following process was used in the experiments reported in this Example. This is the preferred, but not the only, method of generating nanobubbles.

A closed tank (or similar device) maintained at higher than atmospheric pressure is inserted in the regular drinking water line. A recirculation loop that includes a recirculating pump and a nanobubble generator is inserted around the tank. When the recirculating pump is run, the water from the tank flows through the nanobubble generator. At the same time oxygen gas is flowed into the nanobubble generator. The nanobubble generator takes the oxygen gas and creates oxygen nanobubbles in the recirculating water. The water with the oxygen nanobubbles is returned back to the water tank.

The recirculation loop is run for a specified period of time to make sure that the desired characteristics of oxygen nanobubbles are produced. Typically the water in the tank has to be recirculated through the nanobubble generator at least 2 times, more preferably at least 5 times and most preferably at least 20 times. This also creates a relation between flow rate and time for which the recirculation loop is run to create the nanobubbles. For example, if the capacity of the tank is 100 gallons, and the flow rate of the recirculation loop pump is 5 gallons per minute, then the recirculation loop should be run at least 40 minutes.

The tank is typically sized at 1.5 times the maximum amount of water that all the full size birds can drink in one day. The flow rate of the recirculation loop can be varied, and usually depends on the type and design of the nanobubble generator.

The temperature of the water should be maintained within 5 degrees Fahrenheit of the incoming drinking water. Preferably the water containing oxygen-containing nanobubbles should be within 5 F of room temperature. As noted above, preferred temperatures are in the range of 35-120 F, more preferably 50-100 F. This is important because in some cases, the recirculation through the nanobubble generator can cause the temperature of the water to increase. As the temperature of the water increases, its capacity to hold oxygen-containing nanobubbles and dissolved oxygen goes down, negatively affecting its efficacy. Ross 708 (a species of chicken that is usually grown to heavy weights) male chicks from a commercial hatchery were used for the tests. 200 male newly hatched chicks were randomly divided into two equal groups. 100 male chicks were grown while providing them control unoxygenated city drinking water from day 1 (after they hatched) to day 28 of their growth and another 100 male chicks were grown while providing them drinking water containing oxygen-enriched nanobubbles from day 1 to day 28 of their growth. Standard nipple drinkers were used to make drinking water available for both sets of chickens. Both sets of chickens were grown for 28 days. In both cases, the drinking water was always kept pressurized in the water line supplying water to the nipples 20. The chickens were free to drink as much water as they wanted by tapping a nipple 20 to make the water flow. Everything else was maintained the same for both sets of chickens, including feed (other than the water), lighting protocol, and temperatures.

Nanobubbles of oxygen were created in the test drinking water by means of a pressure dissolution-expansion method described above. The city drinking water was pressurized and flowed through a chamber in which oxygen gas was added to the water. The oxygen content of the gas used was 99.99% purity oxygen. The chamber was designed to ensure that all the oxygen that was fed was dissolved into the water. At the outlet of the chamber, the pressure of the water with dissolved oxygen was dropped in a controlled manner so as to allow some of the dissolved oxygen to come out of solution in the form of nanobubbles. This drinking water containing oxygen-enriched nanobubbles was fed to the 100 test birds.

Analysis by NTA of the original drinking water showed that the drinking water contained approximately 129 million bubbles (or particles) per ml of water. After generation of oxygen nanobubbles in the drinking water, a sample of the drinking water with the oxygen-enriched nanobubbles measured by NTA showed 323 million nanobubbles/ml of water. The difference between the two readings showed that approximately 194 million oxygen-enriched nanobubbles were generated per ml of drinking water. Finally, a sample of drinking water with oxygen-enriched nanobubbles was subjected to excessive sodium sulfite solution addition in presence of cobalt catalyst. This addition of sodium sulfite destroyed all the oxygen-enriched nanobubbles after a period of waiting. This sample was then analyzed by NTA which showed approximately 150 million bubbles/particles per ml of drinking water, which is similar to the original 129 million/ml. Thus at least 173 million oxygen-enriched nanobubbles were generated per ml of drinking water and fed to the chicken. These figures are depicted in the bar graphs in FIG. 3.

After 28 days the chickens were evaluated for WB using a trained sensory panel that evaluated the breasts of all 200 chicken from both treatments. In the graph in FIG. 4, a score of 0 indicates no WB was found, a score of 1 indicates mild WB, a score of 2 indicates a medium level of WB, and a score of 3 indicates a very high level of WB. The number of chickens showing a score of 1 or higher than 1 dropped by 72% for the chickens that had been fed water containing oxygen-enriched nanobubbles. There were no differences measured in daily weight gains, final weights, food conversion ratios, mortalities etc. between the two sets of birds.

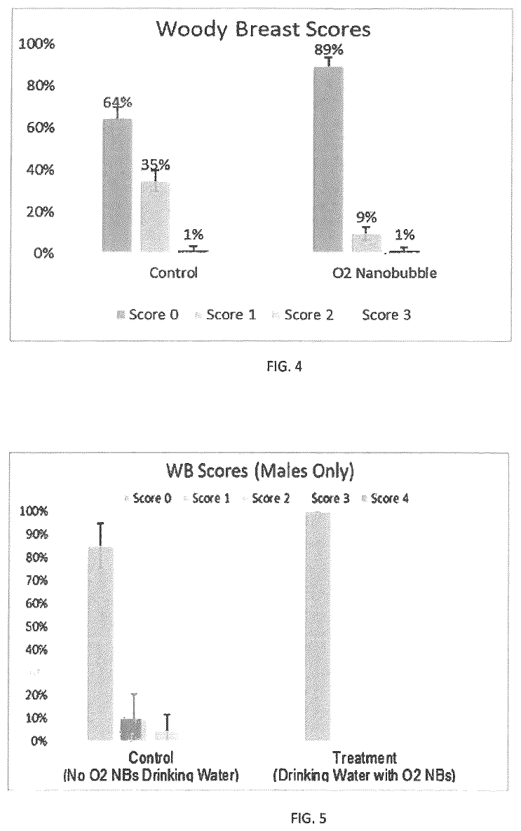

In a second trial the same parameters as above were used, with the exception that the chickens were grown for 42 days instead of 28 days. In this trial the incidence of WB was completely eliminated, as is shown in FIG. 5. There were no differences measured in daily weight gains, final weights, food conversion ratios, mortalities etc. between the two sets of birds.

What is claimed is:

1. A method of reducing or eliminating the incidence of woody breast in a poultry, comprising administering enterally to the poultry water comprising in the water nanobubbles which comprise oxygen.

2. A method according to claim 1 wherein the poultry is a chicken.

3. A method according to claim 2 wherein the water containing said nanobubbles comprises 50 vol. % to 100 vol. % oxygen.

4. A method according to claim 2 wherein the water containing said nanobubbles comprises 90 vol. % to 100 vol. % oxygen,

5. A method according to claim 2 wherein the water containing said nanobubbles comprises at least 99.0 vol. % oxygen.

6. A method according to claim 2 wherein said water containing nanobubbles that is administered enterally to the chicken is maintained at a pressure of up to 25 psig until the chicken ingests it.

7. A method according to claim 2 wherein at least 5%, of all of the water that is administered enterally to the chicken comprises said nanobubbles.

8. A method according to claim 2 wherein 50 vol. % to 100 vol. % of all of the water that is administered enterally to the chicken comprises said nanobubbles.

9. A method according to claim 2 wherein 90 vol. % to 100 vol. % of the water that is made available to the chicken to drink contains said oxygen-containing nanobubbles.

10. A method according to claim 2 wherein the enteral administration to the chicken of said water comprising nanobubbles begins up to 2 weeks after the chicken hatches from its egg.

11. A method according to claim 2 wherein the enteral administration to the chicken of said water comprising nanobubbles begins up to 2 days after the chicken hatches from its egg.

12. A method according to claim 2 wherein the average diameter of said nanobubbles is 10 to 1000 nanometers.

13. A method according to claim 2 wherein the average diameter of said nanobubbles is 10 to 400 nanometers,

14. A method according to claim 2 wherein the average diameter of said nanobubbles is 70 to 130 nanometers.

15. A method according to claim 2 wherein the water administered to the chicken comprises at least 100 million of said nanobubbles per milliliter of water.

16. A method according to claim 2 wherein the water administered to the chicken comprises at least 200 million of said nanobubbles per milliliter of water.

17. A method according to claim 2 wherein the water administered to the chicken comprises at least 500 million of said nanobubbles per milliliter of water.

18. A method according to claim 2 wherein the aggregate amount of oxygen in said water administered to the chicken is 20 to 150 milligrams of oxygen per liter of said water.

19. A method according to claim 2 wherein the aggregate amount of oxygen in said water administered to the chicken is 35 to 100 milligrams of oxygen per liter of said water,

20. A method according to claim 2 wherein the aggregate amount of oxygen in said water administered to the chicken is 60 to 100 milligrams of oxygen per liter of said water.
